(19) United States
(12) Patent Application Publication
(10) Pub. No.: US 2021/0282717 A1
(43) Pub. Date: Sep. 16, 2021

(54) METHODS AND SYSTEMS FOR REDUCING ELECTROCARDIOGRAM ARTIFACTS

(71) Applicant: Duke University, Durham, NC (US)

(72) Inventors: Zak Loring, Durham, NC (US); Jonathan Piccini, Durham, NC (US); Sounok Sen, Durham, NC (US)

(21) Appl. No.: 17/197,206

(22) Filed: Mar. 10, 2021

Related U.S. Application Data
(60) Provisional application No. 62/987,561, filed on Mar. 10, 2020.

(51) Int. Cl. A61B 5/00; G06F 3/14; A61B 5/339; A61B 5/28; A61B 5/318; A61M 60/178; A61M 1/16; A61M 60/139

(52) U.S. Cl. CPC A61B 5/7217; G06F 3/14; A61B 5/725; A61B 5/339; A61M 60/139; A61B 5/318; A61M 60/178; A61M 1/1698; A61B 5/28

(57) ABSTRACT

Methods and systems for reducing electrocardiogram artifacts. In some examples, a system for electrocardiogram monitoring of a patient includes an electrocardiogram receiver configured for receiving one or more signals from one or more electrodes placed on the patient. The system includes a filter configured for filtering the one or more signals to reduce electromagnetic interference produced by an electromechanical support device of the patient, resulting in a filtered signal. The system includes an electrocardiogram display configured for displaying the filtered signal.

### PRIORITY CLAIM

This application claims the benefit of U.S. Provisional Patent Application Ser. No. 62/987,561, filed Mar. 10, 2020, the disclosure of which is incorporated herein by reference in its entirety.

### TECHNICAL FIELD

The subject matter described herein relates generally to electrocardiogram monitoring. More particularly, the subject matter described herein relates to methods and systems for reducing electrocardiogram artifacts.

### BACKGROUND

Ventricular support systems, such as left ventricular assist devices (LVADs) and Impella® pumps, are widely used to assist patients with serious heart conditions. In patients that have these and other types of electromechanical devices, atrial fibrillation that requires electrocardiogram monitoring is also common. However, these types of ventricular support devices are known to cause electromagnetic interference (EMI) on electrocardiogram recordings, which impedes rhythm identification. Further, this EMI may impact screening tests for subcutaneous implantable cardioverters (S-ICDs) and carry a risk for inappropriate ICD therapy delivery. Existing conventional solutions are currently unable to effectively remove all EMI from ECG recordings. Hence, there is a need for improved signal processing in patients using support devices in conjunction with ECGs.

### SUMMARY

This specification describes methods and systems for reducing electrocardiogram artifacts. In some examples, a system for electrocardiogram monitoring of a patient includes an electrocardiogram receiver configured for receiving one or more signals from one or more electrodes placed on the patient. The system includes a filter configured for filtering the one or more signals to reduce electromagnetic interference produced by an electromechanical support device of the patient, resulting in a filtered signal. The system includes an electrocardiogram display configured for displaying the filtered signal.

In some examples, a system for reducing electrocardiogram artifacts includes an adjustable filter configured for filtering one or more signals from one or more electrodes placed on a patient. The system includes a control system configured for selecting one or more filter parameters for the adjustable filter and causing the adjustable filter to reduce electromagnetic interference in the one or more signals produced by an electromechanical support device of the patient.

In some examples, a method for reducing electrocardiogram artifacts includes receiving one or more signals from one or more electrodes placed on a patient. The method includes selecting one or more filter parameters for an adjustable filter. The method includes reducing, by the adjustable filter using the one or more filter parameters, electromagnetic interference in the one or more signals produced by an electromechanical support device of the patient

### BRIEF DESCRIPTION OF THE DRAWINGS

FIG. 1 is a block diagram of an example system for electrocardiogram monitoring;

FIG. 2A is a spectral profile of the ECG data illustrated in FIG. 1;

FIG. 2B is an ECG recording of a patient's heart rhythm;

FIG. 3 is an ECG recording of a patient's heart rhythm after implantation of an LVAD;

FIG. 4 is a spectral profile of the ECG data of FIG. 3;

FIG. 5 is an example of a filtered ECG recording;

FIGS. 6A-6C are spectral profiles of various LVAD devices;

FIG. 7 illustrates a first example signal processing architecture;

FIG. 8 illustrates a second example signal processing architecture;

FIG. 9 illustrates a third example signal processing architecture; and

FIG. 10 is a flow diagram of an example method for reducing electrocardiogram artifacts.

### DETAILED DESCRIPTION

The systems and methods disclosed herein provide a solution to electromagnetic interference (EMI) that can occur on an electrocardiogram (ECG) recording when a patient is simultaneously using a circulatory support device.

The EMI, or “artifact”, is frequently caused by the regular mechanical oscillations of the support device. The system and methods disclosed herein can reduce or remove these types of artifacts by excluding frequency components that are contributed by the circulatory support device, while preserving key cardiac body surface potentials.

For example, patients who have durable LVADs often require ECG monitoring. The LVAD can generate high-frequency noise artifacts on the ECG, which impedes interpretation and rhythm identification.

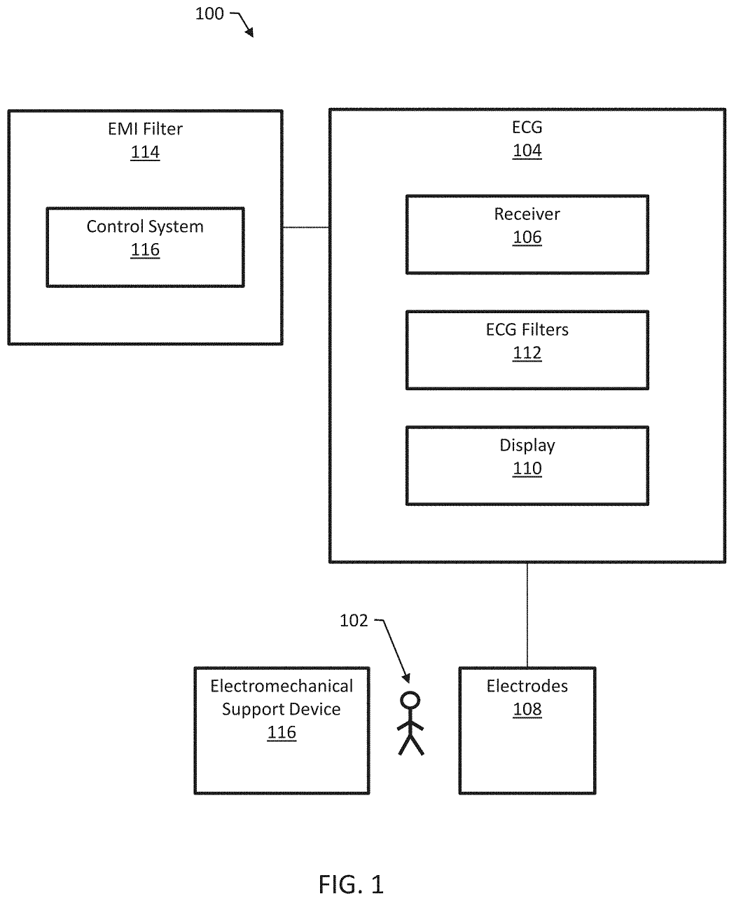

FIG. 1 is a block diagram of an example system 100 for electrocardiogram monitoring of a patient 102. The system 100 includes an ECG 104 including an electrocardiogram receiver 106 configured for receiving one or more signals from one or more electrodes 108 placed on the patient 102. The ECG 104 can also include an electrocardiogram display 110 configured for displaying an ECG signal.

The ECG 104 may be susceptible to interference from non-cardiac sources of electromagnetic activity. The ECG 104 can include filters to reduce or eliminate signal components that arise from external sources and may interfere with ECG interpretation.

For example, the ECG 104 may focus on signals within the 0.04-150 Hz range in order to capture both low frequency components (e.g. details of the ST segment) and high frequency components (e.g. the QRS complex) that are clinically important. This focus is achieved by applying ECG filters 112, e.g., high-pass filters (HPF) and low pass filters (LPF).

HPFs set the lower frequency limit for which signals will be included in the displayed ECG waveform (i.e. only signals higher than this threshold will be included). Conversely, LPFs demarcate the upper frequency limit. Electromagnetic waveforms from power lines can contribute to ECG noise, so an additional “notch” filter may be applied at the typical utility frequency (60 Hz in the Americas and parts of Asia, 50 Hz in much of the rest of the world). The application of HPF, LPF and “notch” filters allows ECG machines to display the signal frequencies within the region of interest while minimizing EMI.

The system 100 also includes an EMI filter 114 configured for filtering the ECG signals to reduce electromagnetic interference produced by a circulatory support device 116 of the patient 102, resulting in a filtered signal. The circulatory support device 116 can be, for example, any appropriate type of device that is applied to the body in a clinical setting to support, assist, and/or monitor the function of the heart or other organs. These devices can include, but are not limited to, left ventricular assist devices (LVADs), extracorporeal membrane oxygenation (ECMO) systems, dialysis systems, percutaneous mechanical circulatory support (e.g., Impella®), intra-aortic balloon pumps, and the like. The circulatory support device 116 can be, for example, a mechanical, electrical, or electromechanical circulatory support device.

In some cases, the circulatory support device 116 includes a motor configured for operating at an oscillation frequency, which can result in EMI at the oscillation frequency or at other frequencies based on the oscillation frequency. The EMI filter 114 is configured for reducing EMI by attenuating frequency components based on the oscillation frequency.

For example, the EMI filter 114 can include a low-pass filter with a cutoff frequency below the oscillation frequency or other frequency components caused by the electromechanical support device 116. In some examples, the EMI filter 114 can include a bandstop filter with a stopband including the oscillation frequency or other frequency components caused by the electromechanical support device 116. A Butterworth filter can be included such that the pass band is maximally flat.

For example, currently available HeartMate 2, HeartMate 3, and HeartWare LVADs were analyzed to identify the LVAD model-specific spectral patterns. The spectral profile of patients with HeartMate 2 and HeartMate 3 LVADs demonstrated a prominent signal at the same frequency of their LVAD's set rotational speed. In HeartMate 3 LVAD patients, two additional peaks were observed at the frequencies equivalent to the LVAD's prespecified “artificial pulse” rotational speeds.

Patients with HeartWare devices demonstrated a prominent signal peak at a frequency equal to double their LVAD's set rotational speed. Applying a LPF to a value below the observed frequency peak from the LVAD significantly improved the legibility and quality of the ECG. Applying a speed-specific bandstop filter to remove the observed LVAD frequency peak also improved the clarity of the ECG without compromising physiological high-frequency signal components.

Each of the three currently available continuous flow LVAD models demonstrated unique signal profile characteristics. The typical rotational speeds of HeartMate 3 LVADs (5,000-6,000 RPM, 83.3-100 Hz) create principal oscillating frequencies in the middle of the typical frequency spectrum of ECGs (0.04-150 Hz). Additionally, the device's “artificial pulse” creates two additional minor frequency peaks in this spectrum.

In some cases, the ECGs from patients with “artificial pulse” devices have the largest amount of electrical artifact. Since publication of the MOMENTUM 3 trial, HeartMate 3 devices have become the predominant choice for LVAD implantation highlighting the clinical importance of this interference. The EMI filter 114 can significantly reduce the interference generated by HeartMate 3 LVADs while preserving high frequency signal components. Adjustment of the LPF is a simple alternative solution that comes at a cost of high frequency signal components, but can be easily done at the time of ECG acquisition or during post-processing in most ECG reading software.

The HeartWare devices were unique in that the principal frequency peak observed in patients with these devices was at a value double the set rotational speed of their LVAD. Like the HeartMate 3, the HeartWare is a magnetically levitated, centrifugal pump implanted in the pericardium. A unique aspect of the HeartWare device is the inclusion of the Lavare cycle, which accelerates and decelerates the pump speed by 200 RPM across a 3 second interval every minute. This infrequent and minor (3.3 Hz) oscillation does not substantially impact the spectral profile of ECGs in these patients. Nonetheless, adjusting the LPF to below the principal frequency peak or applying a “bandstop” filter results in an ECG with less electrical artifact that is easier to interpret.

An increasing number of patients who undergo LVAD implant have a pre-existing subcutaneous implantable defibrillator (S-ICD). Because the primary and secondary sensing vectors of the S-ICD encompass the LVAD, numerous cases of inappropriate S-ICD shocks due to EMI have been frequently reported. This has been more frequently observed in the HeartWare and HeartMate 3 than HeartMate 2, likely due to the high rotational speed and intraperitoneal location of the HeartMate 2.

The second-generation S-ICD incorporated an additional 9 Hz high-pass filter (SMART-PASS) to reduce inappropriate therapy due to oversensing of low-frequency T waves. Incorporation of programmable bandstop filters and/or LPFs into the S-ICD may be applied to patients with LVADs operating at a broader range of frequencies and may help reduce the incidence of inappropriate therapy in the growing population of patients with LVAD and S-ICD.

The EMI filter 114 can implemented using any appropriate technology. For example, the EMI filter 114 can be implemented in software by one or more processors executing a digital signal processing algorithm after the ECG signals have been converted to digital signals. Alternatively, the EMI filter 114 can be implemented as an analog filter circuit and applied to analog electrical signals in the system 100.

The EMI filter 114 can be implemented in any appropriate link of the signal processing chain in the system 100. For example, the EMI filter 114 can be implemented as a stand-alone device or integrated with the ECG 104. The EMI filter 114 can filter the EMI as a pre-ECG step, before the receiver 106 receives the signals, or as a post-ECG step on signal output from the ECG 104. In some examples, the EMI filter 114 is integrated into the ECG 104 and filters the EMI before or after the other ECG filters 112.

In some examples, the EMI filter 114 is adjustable by virtue of a control system 118. The control system 118 is configured for selecting one or more filter parameters (e.g., cutoff frequency or bandstop frequency range) for the EMI filter 114. The control system 118 can be implemented using any appropriate technology. If the EMI filter 114 is implemented in software, then the control system 118 can be implemented as a computer system of one or more processors executing the software.

The control system 118 can be configured for matching the selected filter parameters to particular circulatory support devices of various patients as the system is used for ECG monitoring of different patients with different devices. For example, the control system 118 can include a graphical user interface (GUI) allowing an operator to either input filter parameters directly or allowing an operator to specify the type of circulatory support device. In the case that the operator specifies a type of circulatory support device, the control system 118 can store filter parameters matched to the type of support device and adjust the EMI filter using the stored filter parameters matched to the circulatory support device.

In general, the control system 118 can determine the filter parameters using any appropriate techniques. For example, the control system 118 can determine the type of circulatory support device (e.g., as an alphanumeric string) and then request the filter parameters from a remote computer system over a computer network using a device type identifier. In some cases, the control system 118 includes a wireless receiver and the circulatory support device 116 includes a wireless transmitter, and the circulatory support device 116 is configured to transmit the filter parameters or a device type identifier or both to the control system 118.

The control system 118 can include any suitable components necessary for signal processing, e.g., hardware (including processors, memory devices, displays, user entry devices, etc.), software (including firmware, resident software, micro-code, app etc.) or a combination of hardware and software components. The control system 118 can be configured for processing ECG waveform data, determining the interfering signals, applying filter settings, accepting and transferring the ECG data, providing a user interface, and other similar tasks that will be evident to a person of skill in the art.

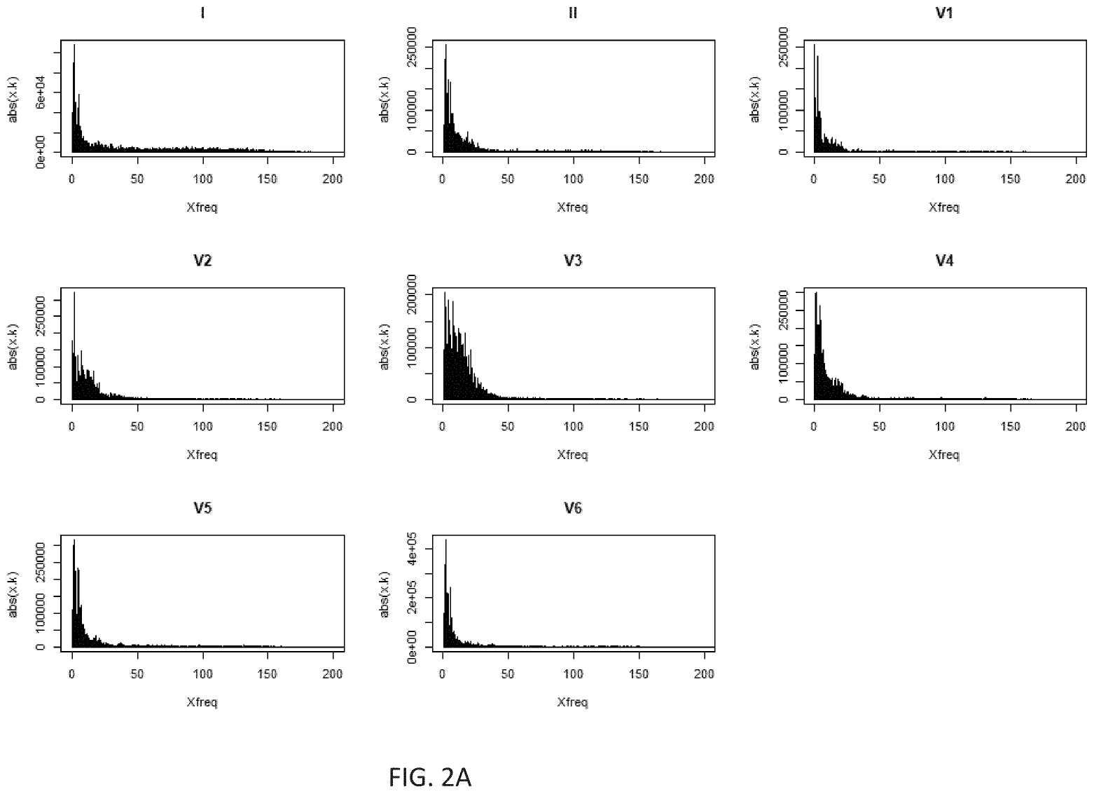

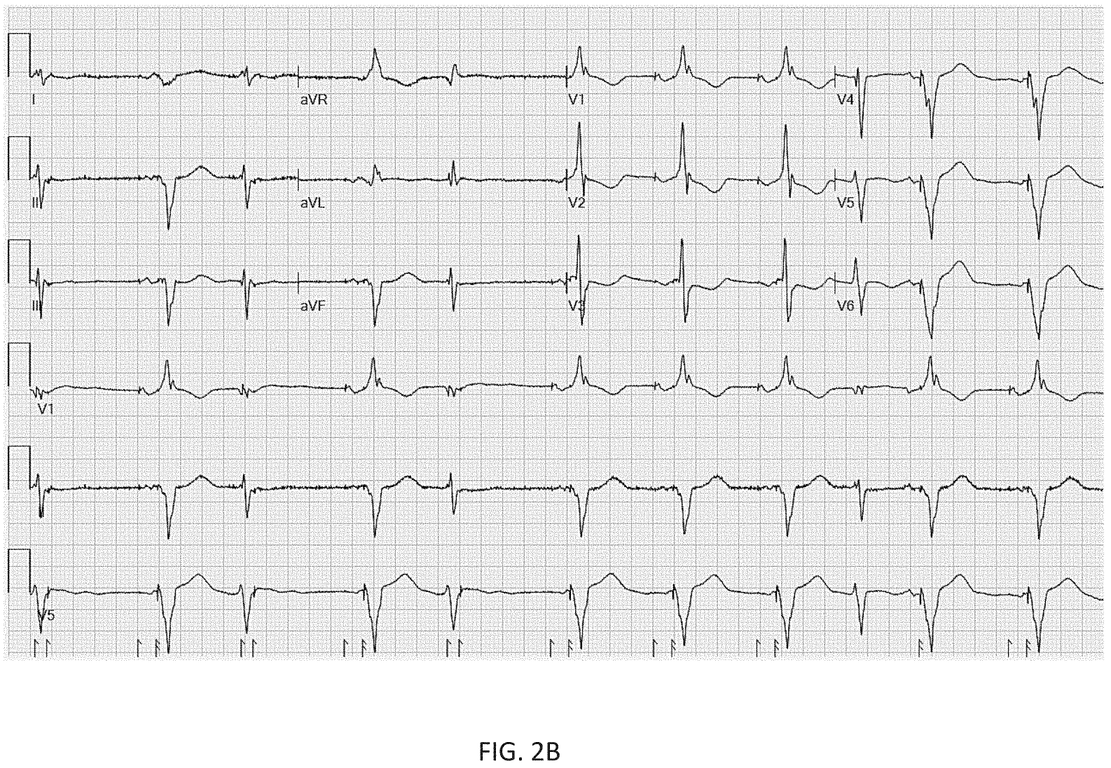

FIGS. 2A and 2B show example data from a patient without a circulatory support device. FIG. 2B shows the output of a 12-lead ECG of the patient. The heart activity is clearly visible, and the patient's ECG does not demonstrate any significant electromagnetic interference. FIG. 2A shows the spectral profile of the same data on the 8 fundamental leads of the ECG (the remaining 4 are derived from these 8 to comprise the 12-lead ECG). A fast Fourier transform (FFT) has been applied to the raw waveform data to display the frequencies that comprise each lead's waveform data. The majority of the signal data is comprised of frequencies below 50 Hz.

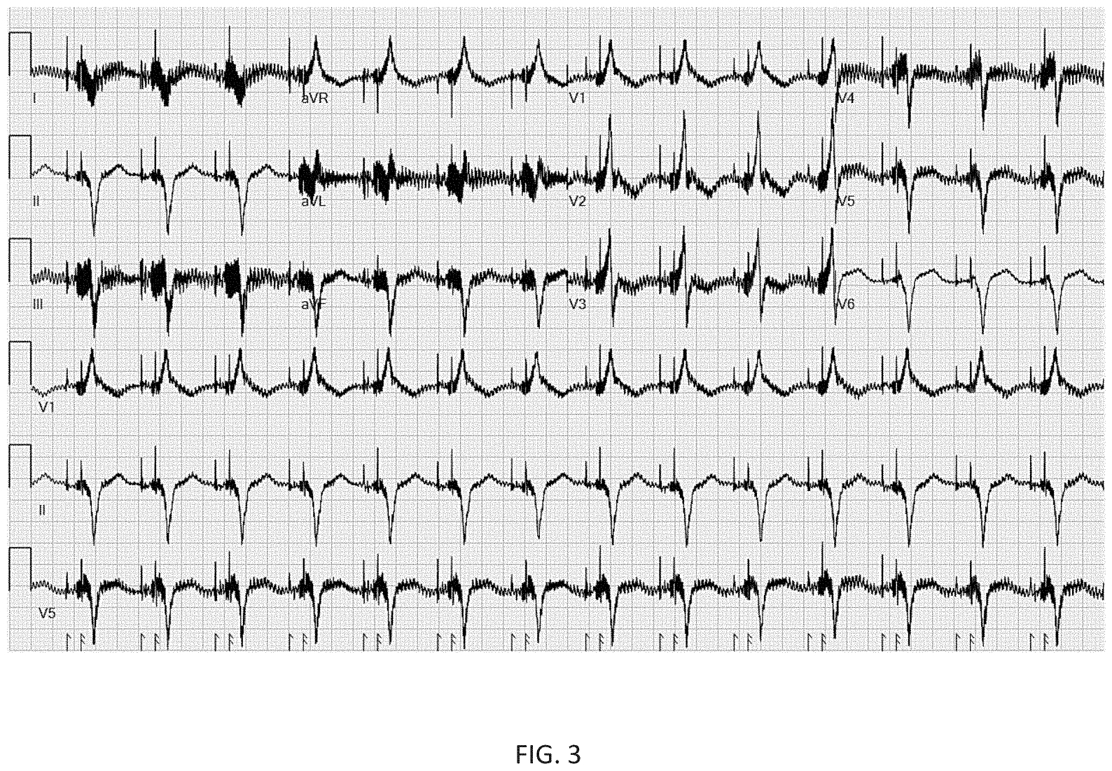

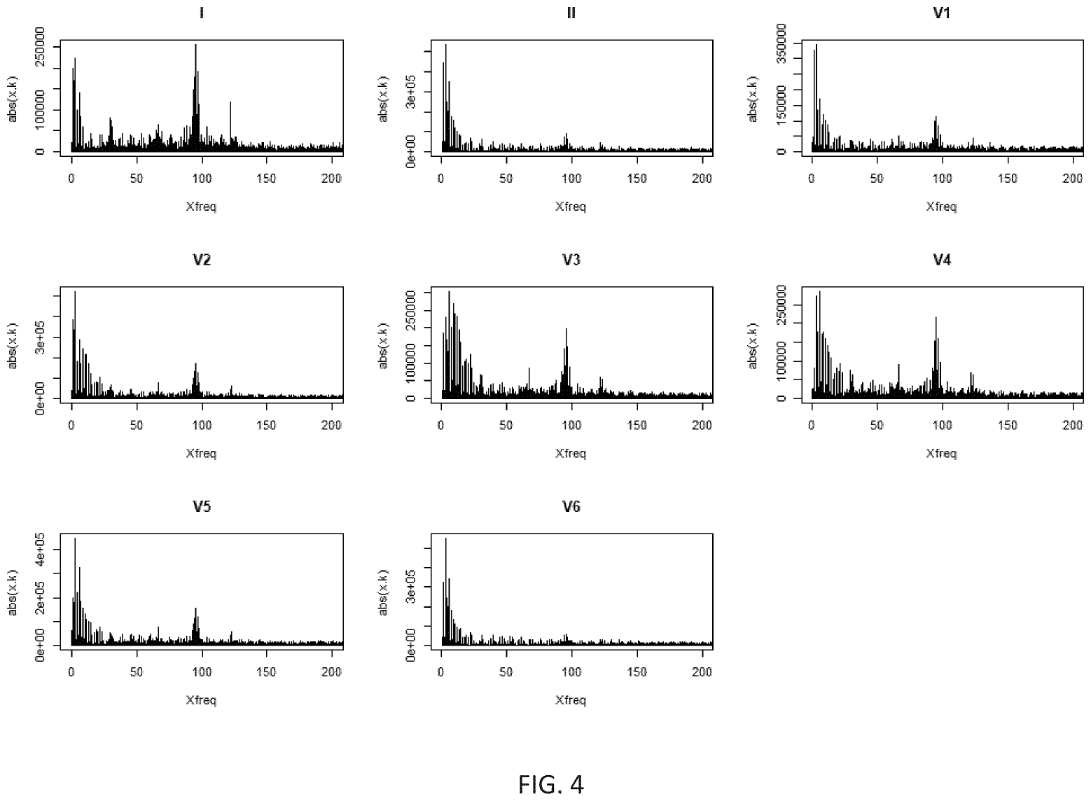

FIG. 3 depicts ECG data from the same patient shortly thereafter, after undergoing implantation of an LVAD. In contrast to the initial ECG, the second ECG after LVAD implantation shows significant EMI artifact. Analysis of the corresponding spectral profile also demonstrates a very different pattern, as seen in FIG. 4. Additional signal peaks are visible at higher frequencies. These signals are contributed by the indwelling LVAD.

Some conventional continuous flow LVADs run at speeds in the range of approximately 2,400-10,000 revolution per minute (RPM). These correspond to oscillating frequencies of about 40-167 Hz, respectively. In the example case shown in FIGS. 2A-2B and FIGS. 3-4, the patient received a Heartmate® 3 LVAD set at a speed of approximately 5700 RPM (95 Hz). This can be seen in FIG. 4, where a frequency peak centered at about 95 Hz is present in all recorded leads.

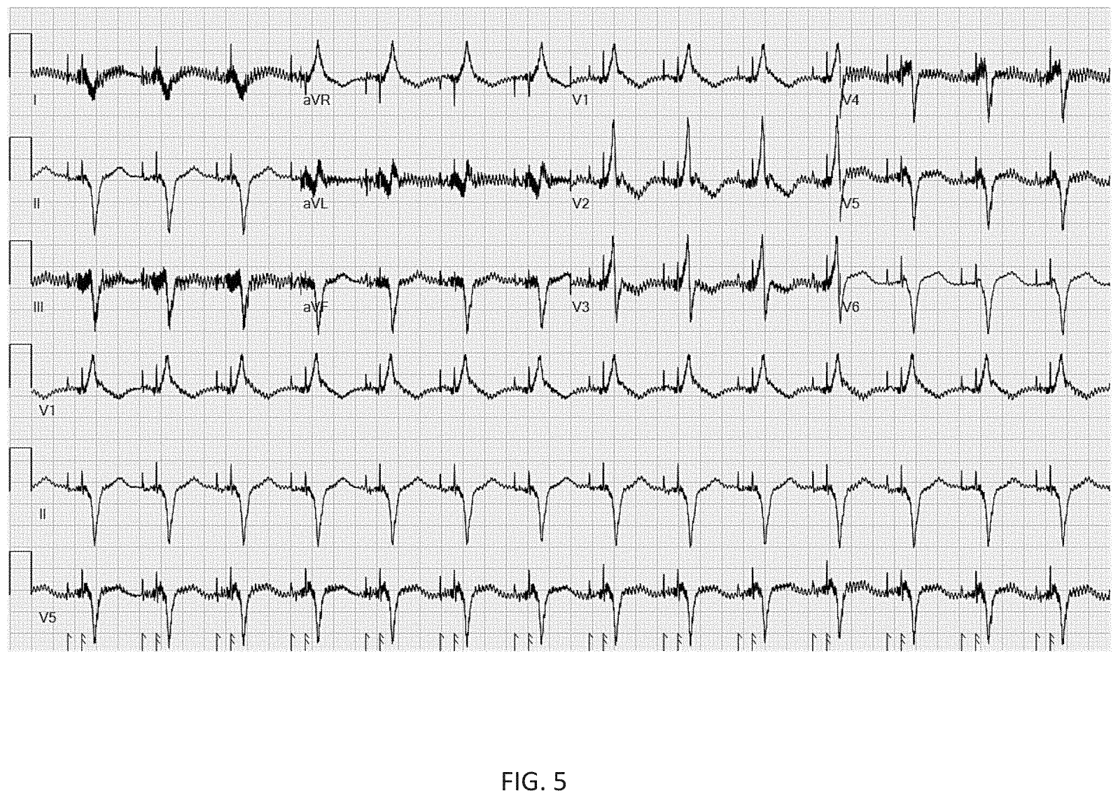

FIG. 5 depicts the same ECG recording as FIG. 3, with a low-pass filter applied to remove EMI as described above with reference to FIG. 1. Here, a LPF with a cutoff frequency of 80 Hz is used. This example illustrates that adjusting the LPF to a value that excludes the speed-related frequency of approximately 95 Hz generates an ECG that has reduced electromagnetic interference artifact and is more easily interpretable.

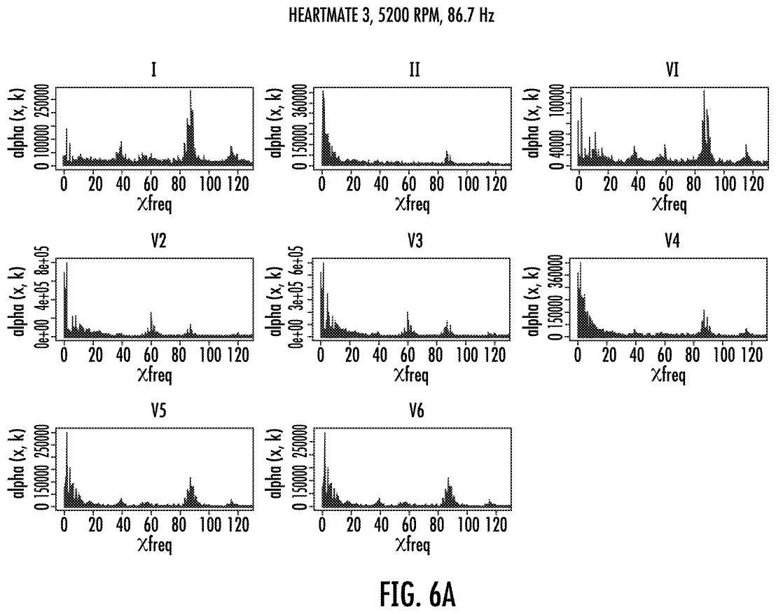

Similar results can be shown for additional example systems. FIGS. 6A-6C depict example spectral profiles for other providers of LVADs operating at different rotational speeds. In each case, spectral peaks occur at the same frequency as the rotational speed. Evaluation of ECGs from patients with HeartMate® 2 and HeartMate® 3 devices set at other speeds consistently demonstrate presence of a signal at the same frequency as the rotational speed of the LVAD.

It is noted that, while adjusting a low-pass filter for patients with circulatory support devices can facilitate rhythm identification, this may also introduce the possibility of discarding high frequency components that carry clinical value. The typical frequencies for the major electrocardiographic waves are typically on the lower end of the bandwidth. However, high frequency components can also contribute to the QRS morphology, particularly in patients with pathology such as scar or hypertrophy.

Thus, in some examples, the systems described in this specification optionally include one or more adjustable filters that excludes a band of frequencies around principal oscillation frequencies contributing to the electromagnetic interference. This allows for a selective, speed-specific exclusion of device-related interference with preservation of the high frequency components of the ECG.

The adjustable filter can be, for example, in the form of a bandstop, band-rejection, or “notch” style filter. Other types of frequency exclusion filters will be apparent to those of skill in the art. The adjustable filter can be configured to exclude a narrow band of signal (e.g., +/−10 Hz) around the principal interference signal. Both the low-pass filter and the adjustable filter can be implemented in any suitable form, such as a hardware device (e.g., a circuit comprising physical components), or as a digital filter (e.g., an algorithm), or a combination of the two.

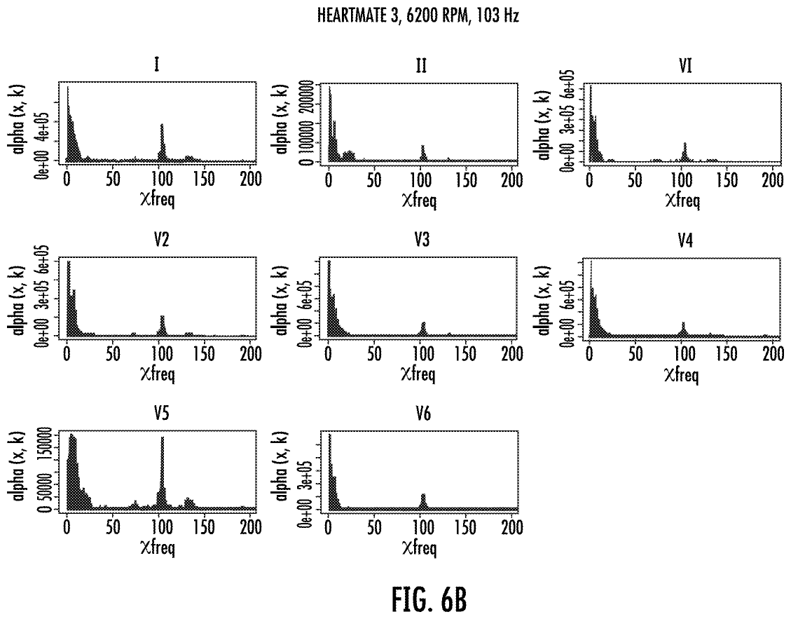

FIGS. 7-9 depict various examples of signal processing architectures for the system 100 of FIG. 1.

FIG. 7 illustrates a first example signal processing architecture. Electrodes are applied to a patient 702 having a circulatory support device. The raw output signals 704 from the electrodes are supplied to an EMI filter 706 that attenuates frequency components that include EMI from the electromechanical support device, resulting in filtered output signals 708. The filtered output signals 708 are supplied to an ECG receiver which, in turn, provides processed output signals 712 for display, storage, or transmission to a remote computer system by a computer network.

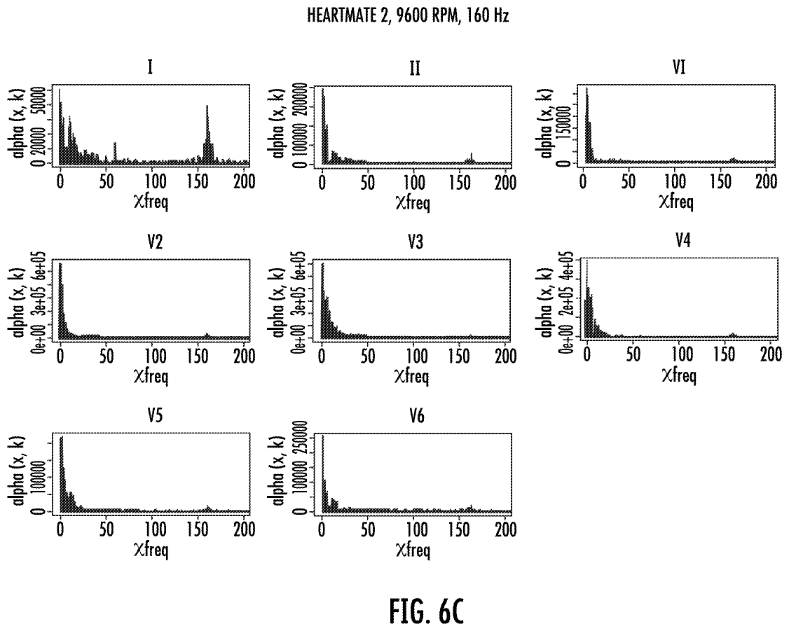

FIG. 8 illustrates a second example signal processing architecture. Electrodes are applied to a patient 702 having an circulatory support device. The raw output signals 704 from the electrodes are supplied to the ECG receiver 710, which includes an integrated EMI filter 706. The filtered output signals 712 are output for display, storage, or transmission to a remote computer system by a computer network.

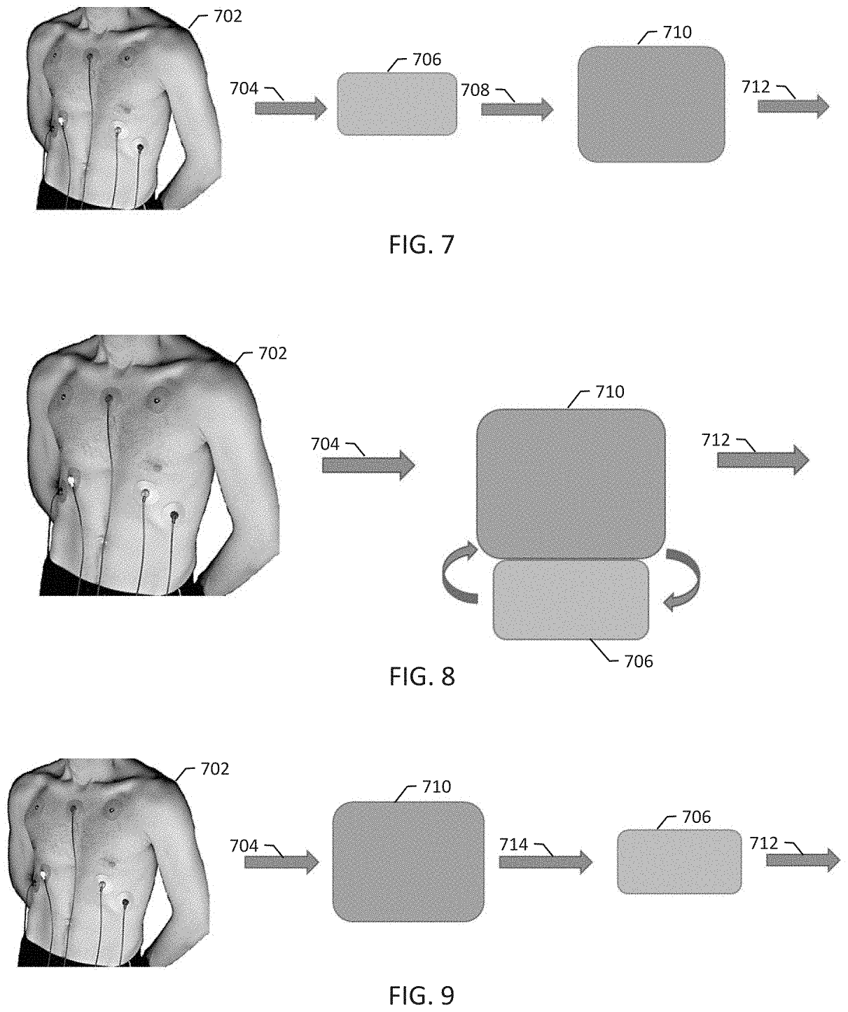

FIG. 9 illustrates a third example signal processing architecture where EMI filtering is performed as a post-processing step applied to the ECG output after recording. Electrodes are applied to a patient 702 having a circulatory support device. The raw output signals 704 from the electrodes are supplied to the ECG receiver 710, which supplies ECG output signals 714 to an EMI filter 706. The filtered output signals 712 are output for display, storage, or transmission to a remote computer system by a computer network

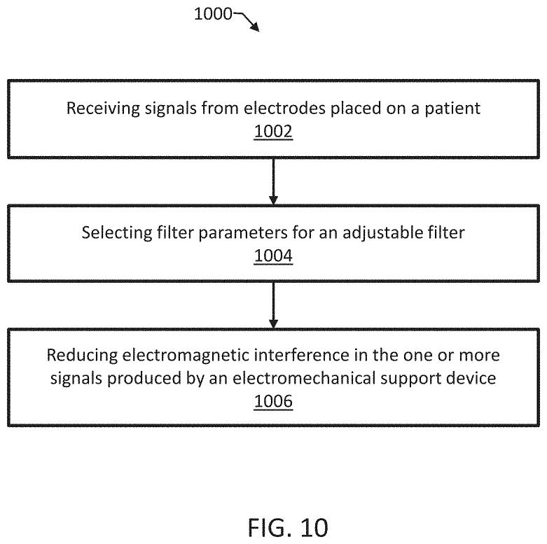

FIG. 10 is a flow diagram of an example method 1000 for reducing electrocardiogram artifacts. The method 1000 can be performed, for example, by the system 100 of FIG. 1. The method 1000 includes receiving one or more signals from one or more electrodes placed on a patient (1002). The method 1000 includes selecting one or more filter parameters for an adjustable filter (1004). The method 1000 includes reducing, by the adjustable filter using the one or more filter parameters, electromagnetic interference in the one or more signals produced by a circulatory support device of the patient (1006).

The control systems described herein can be implemented in hardware, software, firmware, or combinations of hardware, software and/or firmware. In some examples, the control systems described in this specification may be implemented using a non-transitory computer readable medium storing computer executable instructions that when executed by one or more processors of a computer cause the computer to perform operations.

Computer readable media suitable for implementing the control systems described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a control system described in this specification may be located on a single device or computing platform or may be distributed across multiple devices or computing platforms.

Accordingly, while the methods and systems have been described herein in reference to specific embodiments, features, and illustrative embodiments, it will be appreciated that the utility of the subject matter is not thus limited, but rather extends to and encompasses numerous other variations, modifications and alternative embodiments, as will suggest themselves to those of ordinary skill in the field of the present subject matter, based on the disclosure herein.

Various combinations and sub-combinations of the structures and features described herein are contemplated and will be apparent to a skilled person having knowledge of this disclosure. Any of the various features and elements as disclosed herein may be combined with one or more other disclosed features and elements unless indicated to the contrary herein. Correspondingly, the subject matter as hereinafter claimed is intended to be broadly construed and interpreted, as including all such variations, modifications and alternative embodiments, within its scope and including equivalents of the claims.

What is claimed is:

1. A system for electrocardiogram monitoring of a patient, the system comprising:
an electrocardiogram receiver configured for receiving one or more signals from one or more electrodes placed on the patient;
a filter configured for filtering the one or more signals to reduce electromagnetic interference produced by a circulatory support device of the patient, resulting in a filtered signal; and
an electrocardiogram display configured for displaying the filtered signal.

2. The system of claim 1, wherein the circulatory support device comprises a motor configured for operating at an oscillation frequency, and wherein filtering the one or more signals to reduce electromagnetic interference comprises configuring the filter to attenuate one or more frequency components based on the oscillation frequency.

3. The system of claim 2, wherein the filter comprises a low-pass filter with a cutoff frequency below the oscillation frequency.

4. The system of claim 2, wherein the filter comprises a bandstop filter with a stopband including the oscillation frequency.

5. The system of claim 2, wherein the filter comprises a Butterworth filter centered around the oscillation frequency.

6. The system of claim 1, wherein the electromechanical support device comprises a durable left ventricular assist device (LVAD).

7. The system of claim 1, wherein the circulatory support device comprises one of: an extracorporeal membrane oxygenation (ECMO) system, a dialysis system, a percutaneous mechanical circulatory support system, and an intra-aortic balloon pump.

8. A system for reducing electrocardiogram artifacts, the system comprising:
an adjustable filter configured for filtering one or more signals from one or more electrodes placed on a patient; and
a control system configured for selecting one or more filter parameters for the adjustable filter and causing the adjustable filter to reduce electromagnetic interference in the one or more signals produced by a circulatory support device of the patient.

9. The system of claim 8, wherein the circulatory support device comprises a motor configured for operating at an oscillation frequency, and wherein reducing electromagnetic interference comprises configuring the adjustable filter to attenuate one or more frequency components based on the oscillation frequency.

10. The system of claim 9, wherein the adjustable filter comprises a low-pass filter, and wherein selecting one or more filter parameters comprises selecting a cutoff frequency of the low-pass filter below the oscillation frequency.

11. The system of claim 9, wherein the adjustable filter comprises a bandstop filter, and wherein selecting one or more filter parameters comprises selecting a stopband for the bandstop filter including the oscillation frequency.

12. The system of claim 9, wherein the adjustable filter comprises a Butterworth filter, and wherein selecting one or more filter parameters comprises centering the adjustable filter around the oscillation frequency.

13. The system of claim 8, wherein the circulatory support device comprises a durable left ventricular assist device (LVAD).

14. The system of claim 8, wherein the circulatory support device comprises one of: an extracorporeal membrane oxygenation (ECMO) system, a dialysis system, a percutaneous mechanical circulatory support system, and an intra-aortic balloon pump.

15. A method for reducing electrocardiogram artifacts, the method comprising:
receiving one or more signals from one or more electrodes placed on a patient;
selecting one or more filter parameters for an adjustable filter; and
reducing, by the adjustable filter using the one or more filter parameters, electromagnetic interference in the one or more signals produced by a circulatory support device of the patient.

16. The method of claim 15, wherein the circulatory support device comprises a motor configured for operating at an oscillation frequency, and wherein reducing electromagnetic interference comprises configuring the adjustable filter to attenuate one or more frequency components based on the oscillation frequency.

17. The method of claim 16, wherein the adjustable filter comprises a low-pass filter, and wherein selecting one or more filter parameters comprises selecting a cutoff frequency of the low-pass filter below the oscillation frequency

18. The method of claim 16, wherein the adjustable filter comprises a bandstop filter, and wherein selecting one or more filter parameters comprises selecting a stopband for the bandstop filter including the oscillation frequency

19. The method of claim 16, wherein the adjustable filter comprises a Butterworth filter, and wherein selecting one or more filter parameters comprises centering the adjustable filter around the oscillation frequency

20. The method of claim 15, wherein the circulatory support device comprises a durable left ventricular assist device (LVAD).
